(19) United States
(12) Patent Application Publication
(10) Pub. No.: US 2016/0174877 A1
(43) Pub. Date: Jun. 23, 2016

(54) System and Method for Detecting Body Movement

(71) Applicants: David Meltz, Washington, NJ (US); Karen Cohen, Smyrna, GA (US)

(72) Inventors: David Meltz, Washington, NJ (US); Karen Cohen, Smyrna, GA (US)

(21) Appl. No.: 14/977,798

(22) Filed: Dec. 22, 2015

Related U.S. Application Data
(60) Provisional application No. 62/095,251, filed on Dec. 22, 2014.

(51) Int. Cl. A61B 5/11; A61B 5/00; G01P 13/00

(52) U.S. Cl. CPC A61B 5/1116; G01P 13/00; A61B 5/7264; A61B 5/7475; A61B 5/746; A61B 5/486; A61B 5/1114; A61B 5/1121; A61B 5/6801; A61B 2503/10; A61B 2562/0219

(57) ABSTRACT

A system and method for detecting body movement to improve posture and athleticism. The method includes the steps of the logic receiving a user input, such as a body metric input, a physical task, a duration, and an alert type. The processor then determines performance metrics. This can include retrieving an ideal body metric and an ideal motion for the physical task, calculating an optimal performance achievable by the user, and generating an optimal motion for achieving that performance. The logic then receives data from a sensor. This can include detecting a start position and a stop position. Data is recorded and stored in the memory of the system. The logic then transmits an alert, preferably when the stop position is reached. The present system and methods can help improve the quality of life for individuals with neurological disorders and help individuals perform activities where body positioning is important.

### CROSS REFERENCE TO RELATED APPLICATIONS

This application claims the benefit of U.S. Provisional Application No. 62/095,251 filed on Dec. 22, 2014. The above identified patent application is herein incorporated by reference in its entirety to provide continuity of disclosure.

### BACKGROUND OF THE INVENTION

The present invention relates to systems for measuring body movement. More specifically, the present invention relates to systems and methods for monitoring body movement and correcting posture.

Individuals with poor posture can experience major difficulties in performing physically demanding tasks later in life. Maintaining correct posture is important to preventing and healing back injuries. Improper posture can cause stress, pressure, and potential damage to the lower back. Neck and shoulder pain may also result from improper posture. Proper posture is especially important in society today, where the majority of individuals are spending more and more time in front of a computer. This is significantly apparent in the workplace where musculoskeletal injuries are extremely costly to employers. Poor body positioning during work, such as slouching in a chair or curving the spine forward while standing for long hours, can lead to poor employee health and morale. Ultimately, workplaces will experience less productivity and higher overall costs.

Although numerous back injuries may be caused by incorrect posture, good posture can also reduce the symptoms of conditions such as osteoporosis and scoliosis. Similarly, proper posture may help improve the quality of life for individuals who suffer from neurological disorders such as Parkinson's disease and other neural-muscular problems. With good posture, breathing is easier and muscle movement requires less effort.

Proper posture can also improve athletic performance. Further, making adjustments to body movement angles during athletic activities can improve the power and efficiency of an athlete. For example, determining an optimal angle at which a user may swing a baseball bat can allow the user to train himself to generate most power possible thereby hitting the ball as far as possible. Therefore, there is a need for a device that informs individuals when they are not maintaining proper posture or informs individuals when they have reached a proper or optimal body position.

### SUMMARY OF THE INVENTION

In view of the foregoing disadvantages inherent in the known types of posture aids now present in the prior art, the present invention provides a system and method for detecting, monitoring, and recording body movement wherein the user may receive alerts when a wearable sensor has detected a change in body position. The present system comprises a terminal housing a processor, memory, and logic; wherein the terminal is a wearable mobile device. The wearable mobile device can be attached via a strap to a body area the user desires to monitor. The terminal further comprises one or more sensors such as a gyroscope, clinometer, and an accelerometer. The terminal additionally comprises a speaker, headphone jack, LED light, and vibration motor wherein the logic transmits an alert to the terminal that is emitted through the speaker, headphone jack, LED light, or vibration motor when the user reaches a stop position or exhibits an incorrect posture.

The method comprises the first step of receiving user input. User input can comprise a body metric, a physical task, a duration, or an alert type. A body metric can be measurements such as the height or weight of a user or a length of the arm from the shoulder to the wrist. A physical task is any type of exercise or desired body movement, such as a golf swing. A duration can comprise the number of repetitions of the physical task or a length of time. An alert type can comprise a vibratory signal, a flash of an LED light, an auditory signal, or a combination of the three.

At the next step, the logic determines performance metrics. In an embodiment wherein a user input is a physical task, the logic retrieves an ideal body metric and an ideal body motion for that physical task. The processor then calculates the optimal performance achievable by a user having the body metrics inputted into the system. The processor then generates an optimal motion for a user having those body metrics to execute in order to achieve the optimal performance.

Next, the sensors begin to monitor the body movements of a body area wherein the sensor is attached. Data collected from the sensor is transmitted to the logic wherein the processor continuously monitors each incident of specific body movement. In an alternative embodiment, the processor monitors the body movement until a start position is reached wherein the data begins to then be recorded and transmitted to the memory for storage. In this embodiment, the recording continues until a stop position is reached. Once the stop position is reached, an alert is transmitted to the terminal where it is emitted in the form of the alert type inputted.

In another embodiment preferably monitoring posture, a sensor is attached to a body area such as a chest or waist. The logic receives an angle of incidence input defining a threshold angle that the user seeks to avoid. The sensor then transmits data to the logic wherein the processor calculates the angle of incidence. If the angle of incidence calculated extends beyond the threshold angle, an incorrect posture is detected. An alert is then transmitted to a terminal. This alert informs the user that an incorrect posture has been reached thereby allowing the user to correct his or her posture.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of a detail view of an embodiment of the system for detecting body movement in use.

FIG. 2 shows a flowchart illustrating an embodiment of a method for monitoring body movement.

FIG. 3 shows a flowchart illustrating an embodiment of a method for monitoring body movement.

FIG. 4 shows a flowchart illustrating an alternative embodiment of a method for monitoring body movement.

### DETAILED DESCRIPTION OF THE INVENTION

Reference is made herein to the attached drawings. Like reference numerals are used throughout the drawings to depict like or similar elements of the system and method for detecting body movement. For the purposes of presenting a brief and clear description of the present invention, the preferred embodiment will be discussed as a system and method for detecting body movements. The figures are intended for representative purposes only and should not be considered to be limiting in any respect.

As used herein, “processor” refers to one or more devices, circuits, and/or cores configured to process data, such as a set of steps according to a computer program. Unless stated otherwise, a component such as a processor or interface described as being configured to perform a task includes both components temporarily configured to perform a task at a specified time and components manufactured to perform a task. As used herein, “logic” refers to (i) logic implemented as computer instructions and/or data within one or more computer processes and/or (ii) logic implemented in electric circuitry. As used herein, “computer-readable medium” excludes any transitory signals, but includes any non-transitory data storage circuitry, e.g., buffers, cache, and queues, within transceivers of transitory signals. Unless stated otherwise, a “terminal” refers to a desktop computer, a smartphone, or any other similar mobile device. The terminal houses the processor, logic, and memory.

According to some embodiments, the operations, techniques, and/or components described herein can be implemented as (i) a special-purpose computing device having specialized hardware and a logic hardwired into the computing device to persistently perform the disclosed operations and/or techniques or (ii) a logic that is implementable on an electronic device having a general purpose hardware processor to execute the logic and a computer-readable medium, e.g. a memory, wherein implementation of the logic by the processor on the electronic device provides the electronic device with the function of a special-purpose computing device. The database is connected to the logic via a connection medium. The connection medium includes a wired or wireless connection to the database via a telecommunications network, e.g., the Internet, or via a communications protocol, e.g. Bluetooth, that is not network-based.

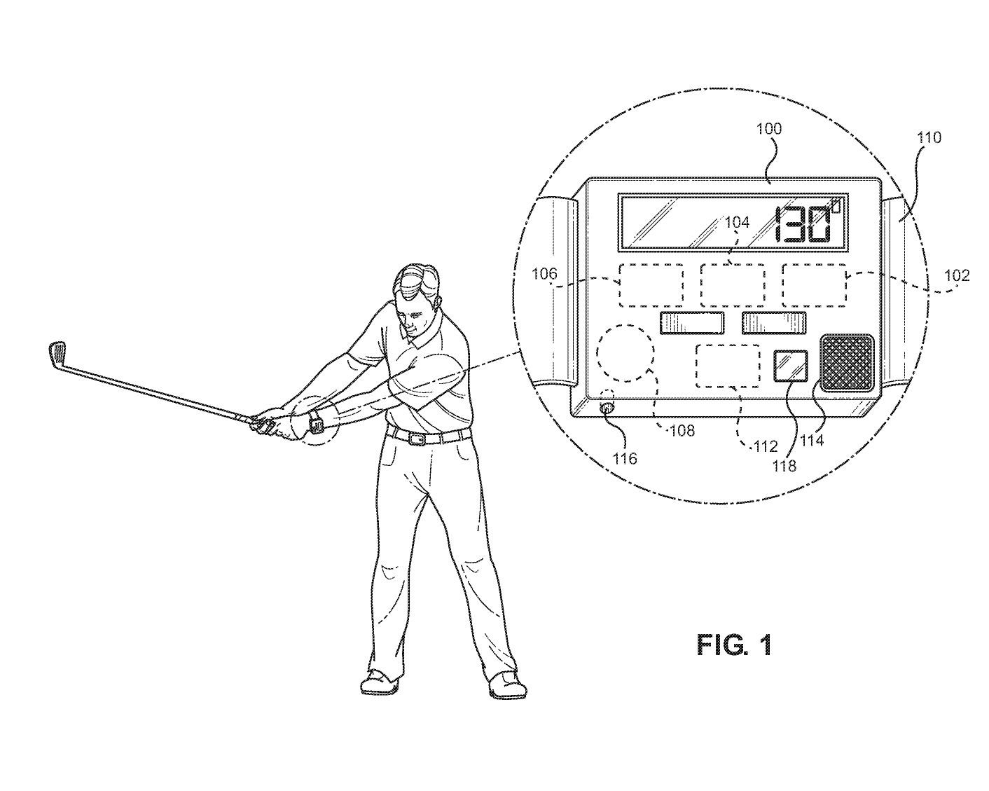

Referring now to FIG. 1, there is shown a detail view of an embodiment of the system for detecting body movement in use. In the depicted embodiment, a terminal of the system is a wearable mobile device 100 comprising a processor 102, a logic 104, and a memory 106 operably connected therein. In an alternative embodiment, the terminal is a desktop computer that wirelessly communicates a wearable mobile device. In the depicted embodiment, the mobile device 100 further comprises a sensor 108 operably connected thereto. The sensor can be one or more accelerometers, clinometers, or gyroscopes. In the depicted embodiment, the mobile device 100 is connected to a strap 110 secured around the wrist of the user. However, the strap 110 can be secured around any body area the user desires to monitor. In alternative embodiments, it is contemplated that the sensor can be pinned to a user's garment, inserted into a user's pockets, attached to a user's glasses via a clip, contained within an ear cuff, or any other suitable means.

In the illustrated embodiment, the mobile device 100 further comprises an alert device wherein the alert device comprises a vibration motor in some embodiments, an LED light, or a speaker system in other embodiments. The vibration motor 112 causes the terminal to vibrate thereby transmitting a vibratory alert to the user when the user reaches a stop position. The speaker system comprising a speaker 114 and a headphone jack 116 transmit an auditory alert when the user reaches a stop position. The LED light 118 will emit a flash when the user reaches a stop position. The LED light, speaker system, and vibration motor can be used separately or in conjunction with each other.

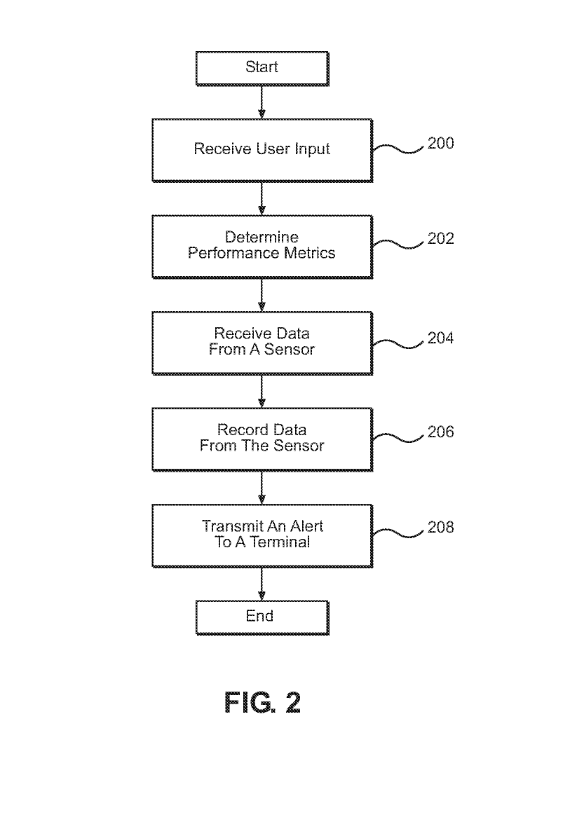

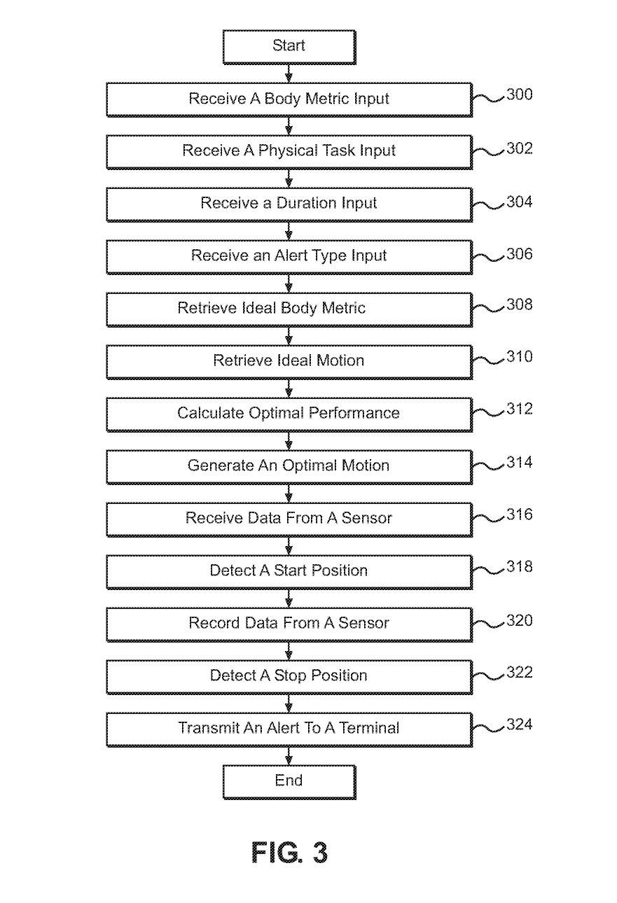

Referring now to FIGS. 2 and 3, there are shown flowcharts illustrating embodiments of a method for monitoring body movement. At 200, the logic receives one or user inputs. A user input can include a body metric 300, a physical task 302, duration 304, or an alert type 306. A body metric 300 can include, but is not limited to height, weight, arm length, and gender. In the preferred embodiment, a user inputs one or more of his or her own body metrics on the terminal. Once the logic receives the body metrics of a user, the body metrics are stored in a database on the memory. Inputting body metrics allows the processor to generate more accurate performance metric customized for the specific user.

A user input can also comprise a physical task 302. A physical task 302, in one embodiment, is a pre-loaded exercise stored in the database. For example, a user can choose from pre-loaded exercises such as a tennis serve, baseball swing, or golf swing. In another embodiment, the physical task is a saved exercise stored in the database. A saved exercise is a physical task previously selected by the user.

In one embodiment, a duration 304 is a number of repetitions of the physical task. For example, the user could enter thirty golf swings. In another embodiment, the duration is a length of time, such as twenty minutes or three hours. In this embodiment, the length of time selected could also be a continuous designation wherein the body movement is monitored until the logic receives a termination input entered by the user.

In another embodiment for this method, a user input comprises an alert type 306. In one embodiment, the alert type 306 is an auditory signal, such as music, an alarm, a beep, or a buzzer. In another embodiment, the alert type 306 is a vibratory signal. Further, it is contemplated that a user can specify the length and pattern of vibrations of the vibratory signal. In an alternative embodiment, the alert type 306 is a flash from an LED light. In another embodiment, the alert type 306 is a combination of the auditory signal, the flash from the LED light, and the vibratory signal.

At 202, the processor determines performance metrics. In one embodiment, the processor retrieves ideal body metrics 308 and ideal motions 310 that correspond to the physical task 302. The ideal body metrics 308 and ideal motions 310 for a physical task 302 are stored within the database. For example, the database will have the ideal body metrics and accompanying ideal motions in order for a male to throw a baseball at the fastest speed achievable. The ideal body metrics and ideal motions for each physical task are pre-programmed into the database.

In one embodiment, once the processor has retrieved the ideal body metrics and ideal motions for a physical task, the processor calculates an optimal performance 312 for a user. In this embodiment, the optimal performance is calculated by differentiating the dimensions, physics, and biomechanical characteristics of a user from the ideal body metrics and ideal motions. For example, if for a six-foot tall man, the ideal arm length is thirty-four inches for achieving the fastest speed of baseball pitch and the user's arm length is thirty-three and one-quarter inches, the processor will take into account the arm length differential when determining how fast the user could pitch a baseball.

In an embodiment, the processor then generates one or more optimal motions 314 to be carried out by a user in order to achieve optimal performance of a physical task. Once the optimal performance for a user has been calculated, the processor generates optimal motions that function as instructions describing how the user can achieve the optimal performance. For example, the optimal motion could be rotating the shoulder ninety degrees when throwing the baseball in order to achieve the fastest possible baseball pitch for that particular user.

At 204, data is received from a sensor thereby monitoring the body movement of a user. In one embodiment, the terminal of the system is a wearable device, as shown in FIG. 1. The wearable device comprises one or more sensors for detecting body movement. A sensor could be a clinometer, an accelerometer, or a gyroscope. A clinometer will measure a tilt angle of a body area. The tilt angle is measured when the clinometer generates an artificial horizon and measures the angular tilt of a body area with respect to this horizon. An accelerometer will measure the acceleration of a body area. A gyroscope can measure both the orientation and rotation of a body area. The device can comprise one of these three types of sensors or any number and combination of them. The sensors transmit these measurements to the logic in the form of data.

In various embodiments, receiving data from the sensor 204 comprises detecting a start position 316. In this embodiment, no data is recorded or stored on the memory of the system until the system detects that the user's body movement has reached a start position. For example, if the physical task is a pre-loaded exercise such as a tennis serve, the user's body movement will not reach the start position until the user raises his or her arm to initiate the serve. This allows the user to program the system and move around without recording copious amounts of irrelevant data.

At 206, the system records the data transmitted by the sensors. Once the logic receives data from the sensors, the data is recorded when it is transmitted to the memory of the system. In the embodiment shown in FIG. 3 wherein a start position is detected 316, data from the sensor is not recorded until the start position is reached. Further, in this embodiment, data is recorded until the system detects a stop position 318. Based on the data transmitted by the sensor, the system can detect when the user's body movement has reached the stop position and the data will no longer be recorded.

At 208, the system transmits an alert to the terminal. In the embodiment depicted in FIG. 3, the alert transmitted 318 to the terminal corresponds to the alert type 306. For example, if the alert type is a vibratory signal, a device comprising the terminal such as that shown in FIG. 1, will vibrate. In the embodiment shown in FIG. 3, the alert provides a notification to the user that the stop position has been reached. In another example wherein the alert is an auditory signal, such as a beep, the auditory signal is emitted. In one embodiment, the auditory signal is emitted through a speaker on the terminal. In another embodiment, the auditory signal is emitted through a headphone jack on the terminal when the user's body movement has reached the stop position. After an alert has been received, the user can review the data stored on the memory.

For example, a user can secure a mobile device comprising a sensor, such as that shown in FIG. 1, around his wrist. The user then enters his body metrics, such as height and weight. Next, the user selects the physical task of a golf swing. The user may select a duration such as one repetition of the golf swing and an alert type such as an auditory signal. The processor retrieves the ideal body metrics and ideal motion for achieving a powerful golf swing. This could be a height of six feet, a weight of a hundred and eighty pounds, a backswing of two-hundred and thirty-seven degrees, and a club speed of eighty miles per hour. The processor then calculates an optimal performance for someone of the user's height and weight by differentiating the ideal body metrics and the user's body metrics. The processor then generates an ideal motion designed to give this specific user the most powerful golf swing achievable for someone with his body metrics. The ideal motion comprises a start position and a stop position. The sensor on the user's wrist detects the start position when the user raises the club and detects when the user follows through with the swing to a stop position. Data from the swing is transmitted and stored on the memory of the mobile device. When the stop position has been reached, an auditory alert, such as a beep, is transmitted through a speaker on the mobile device, or through headphones attached to the mobile device. The user can then review the data stored on the mobile device.

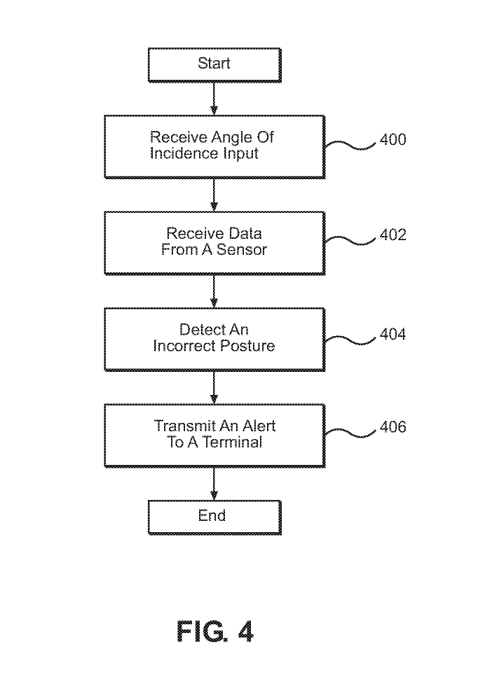

Referring now to FIG. 4, there is shown a flowchart illustrating an alternative embodiment of a method for monitoring body movement. The embodiment shown in FIG. 4 is preferably a method for monitoring posture. In the depicted embodiment, the system receives an angle of incidence input 400. The angle of incidence is defined as the angle a ray makes with a perpendicular to the surface at the point of incidence. For example, if a user is sitting in a chair with a straight back the angle of incidence is zero because his or her body is perpendicular to a surface, such as a seat or a floor. However, if the user begins to lean forward thereby exhibiting poor posture, an angle of incidence is created between the user's back and the perpendicular to the surface. The angle of incidence input 400 is a threshold angle that the user seeks to avoid.

At 402, data is received from a sensor. To monitor and correct posture, a sensor can be attached to a body area of a user, such as around the chest or waist. Similar to the embodiments shown in FIGS. 2 and 3, sensors such as a clinometer, an accelerometer, or a gyroscope transmit data to the logic. Once the logic receives data from the sensor, the processor determines the angle of incidence therefrom. At 404, when the user's body area, such as the chest, forms an angle of incidence that extends beyond the threshold angle, the logic detects this as an incorrect posture. At 406, an alert is transmitted to the terminal thereby notifying the user of the incorrect posture. The user can correct his or her posture based on this feedback.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A computer system for detecting body movement, comprising:
a processor;
a non-transitory computer readable medium operatively connected to the processor;
a logic stored in the non-transitory computer readable medium that, when executed by the processor, causes the computer system to perform a method, the method comprising the steps of:
receiving a user input regarding a physical task to be completed;
determining performance metrics;
receiving data from a sensor positioned on a body area of a user;
recording data from the sensor as the user performs the physical task; and
transmitting an alert to a terminal.

2) The method of claim 1, wherein a user input is a body metric of the user.

3) The method of claim 1, wherein a user input is a physical task to be performed by the user.

4) The method of claim 1, wherein a user input is a duration of time through which the physical task is to be performed.

5) The method of claim 1, wherein a user input is an alert type.

6) The method of claim 1, wherein the step of determining performance metrics comprises the step of retrieving an ideal body metric from a database.

7) The method of claim 1, wherein the step of determining performance metrics comprises the step of retrieving an ideal body motion from a database.

8) The method of claim 6, wherein the step of determining performance metrics comprises calculating an optimal performance based upon differences between the user input and the ideal body metric.

9) The method of claim 1, wherein the step of determining performance metrics comprises generating an optimal motion.

10) The method of claim 1, wherein the sensor is configured to measure acceleration of a body area on which the sensor is mounted.

11) The method of claim 1, wherein the sensor is configured to measure a tilt angle of a body area.

12) The method of claim 1, wherein the sensor is configured to measure an orientation and a rotation of a body area.

13) The method of claim 1, wherein the step of receiving data from a sensor further comprises the step of detecting a start position for the physical task.

14) The method of claim 1, wherein the step of receiving data from a sensor further comprises the step of detecting a stop position for the physical task.
